(19) United States
(12) Patent Application Publication
(10) Pub. No.: US 2021/0015719 A1
(43) Pub. Date: Jan. 21, 2021

(54) STABLE EMULSIFIED GEL COMPOSITION HAVING A HIGH CONCENTRATION OF ACTIVE COMPONENTS

(71) Applicant: L'OREAL, Paris (FR)

(72) Inventors: Gloria LU, Rahway, NJ (US); Patricia BRIEVA, Manalapan, NJ (US); Donna McCANN, Oxford, NJ (US); Nannan CHEN, Princeton, NJ (US); Lauren E. MANNING, Hoboken, NJ (US)

(73) Assignee: L'OREAL, Paris (FR)

(21) Appl. No.: 17/064,348

(22) Filed: Oct. 6, 2020

Related U.S. Application Data
(63) Continuation-in-part of application No. 15/614,058, filed on Jun. 5, 2017.

(51) Int. Cl. A61K 8/04; A61K 8/73; A61K 8/60; A61K 8/9794; A61K 8/37; A61K 8/86; A61K 8/891; A61Q 5/00; A61Q 19/00

(52) U.S. Cl. CPC A61K 8/042; A61K 8/735; A61K 8/60; A61K 8/9794; A61K 8/37; A61K 2800/52; A61K 8/891; A61Q 5/00; A61Q 19/00; A61K 2800/10; A61K 8/86

(57) ABSTRACT

An emulsified gel composition having an emulsified gel system having an emulsifier and a hyaluronic acid delivery component. The hyaluronic acid delivery component includes a combination of active ingredients. The combination of active ingredients includes each of a hyaluronic acid, a hyaluronidase inhibitor, and hydroxypropyl tetrahydropyrantriol. The composition is stable and has a pleasing tactile feel. A method for cosmetic treatment of keratinous tissues is also disclosed.

### RELATED APPLICATION

The present application is a continuation in part of and claims priority to U.S. patent application Ser. No. 15/614,058 entitled A STABLE EMULSIFIED GEL COMPOSITION HAVING A HIGH CONCENTRATION OF ACTIVE COMPONENTS, which was filed on Jun. 5, 2017, the entirety of which is incorporated herein by reference.

### FIELD OF THE INVENTION

The present invention is directed to a stable skin care composition having a high concentration of active components. More specifically, the present invention is directed to a stable skin care composition having a hyaluronic acid delivery component including a hyaluronic acid, a hyaluronidase inhibitor, and hydroxypropyl tetrahydropyrantriol.

### BACKGROUND OF THE INVENTION

Oil-in-water (O/W) emulsions are well known in the field of cosmetics and dermatology, in particular for the preparation of cosmetic products, such as milks, creams, tonics, serums or toilet waters. These emulsions, that consist of an oil phase (or lipophilic phase) dispersed in an aqueous phase, have an external aqueous phase and are therefore products that are more pleasant to use because of the feeling of freshness that they provide. However, they have the drawback of relatively lacking stability when the amount of oil present is too great. Now, for some applications, it is advantageous to have a large amount of oils since the oils provide comfort for the skin, nourish it, and can also remove makeup from it when these oils have makeup-removing properties.

Hyaluronic acid is a natural mucopolysaccharide formed of alternating units of D-glucuronic acid and N-acetylglucosamine in a linear chain. Hyaluronic acid is material that is present in human tissues, such as skin and ligaments. In addition, hyaluronic acid serves as a connective tissue supporter and water retaining substance. Hyaluronic acid is found both the dermis and the epidermis of the skin, where it has a protective, structure stabilizing and shock-absorbing function. Hyaluronic acid has the ability to penetrate the skin and provide moisturizing and viscoelastic properties to the skin making the skin softened and restoring its elasticity, which is capable of providing an antiwrinkle effect.

It is difficult to produce fine O/W emulsions containing a large concentration of active ingredients, particularly hyaluronic acid, since such emulsions have a tendency to destabilize. This destabilization results in coalescence and separation of the aqueous and oily phases with release of the oil. In order to improve the stability of these emulsions, the concentration of emulsifiers can be increased; however, a high concentration of emulsifiers can result in a rough, clingy or sticky feel, and in problems of innocuity with respect to the skin, the eyes and the scalp. Therefore, effective delivery and absorption of hyaluronic acid in skin with cosmetic compositions is difficult to achieve.

There remains a need to provide a composition, including skin care and sunscreen compositions, capable of stably carrying high loading of actives, including hyaluronic acid, which is also tactilely pleasing to consumers upon application.

### BRIEF SUMMARY OF THE INVENTION

In an exemplary embodiment, a composition in the form of a stable skin care composition including an emulsified gel composition. The gel composition includes an emulsified gel system having an emulsifier and a hyaluronic acid delivery component. The hyaluronic acid delivery component includes a combination of active ingredients. The combination of active ingredients includes each of a hyaluronic acid, a hyaluronidase inhibitor, and hydroxypropyl tetrahydropyrantriol. The composition is stable and has a pleasing tactile feel.

In an exemplary embodiment, a composition in the form of a stable skin care composition including an emulsified gel composition. The gel composition includes an emulsified gel system having an emulsifier and a hyaluronic acid delivery component. The hyaluronic acid delivery component includes a combination of active ingredients. The combination of active ingredients includes each of from about 0.1 to about 1.4% by weight of a hyaluronic acid, from about 0.1 to about 3.0% by weight of a hyaluronidase inhibitor, and from about 1.0% to about 12.0% by weight hydroxypropyl tetrahydropyrantriol. The composition is stable and has a pleasing tactile feel.

The present disclosure is also directed to a method for cosmetic treatment of keratinous tissues by applying the above-disclosed composition onto a surface of the keratinous tissue.

Other features and advantages of the present invention will be apparent from the following more detailed description of the preferred embodiment which illustrates, by way of example, the principles of the invention.

### DETAILED DESCRIPTION OF THE INVENTION

All numbers expressing quantities of ingredients and/or reaction conditions are to be understood as being modified in all instances by the term “about”, unless otherwise indicated.

“Keratinous tissue,” as used herein, includes, but is not limited to, skin, hair, and nails.

“Homogenous” means having the visual appearance of being substantially uniform throughout, i.e., visually appears as a single phase emulsion.

It has been surprisingly and unexpectedly discovered by the inventors that the combination of hyaluronic acid, hyaluronidase inhibitor, and hydroxypropyl tetrahydropyrantriol in specific amounts in an emulsified gel provides exceptional stability and skin absorption properties, while retaining a pleasing tactile sensation.

The composition according to the present disclosure includes an emulsified gel system comprising an emulsifier and a hyaluronic acid delivery component.

Hyaluronic Acid Delivery Component

The hyaluronic acid delivery component includes a combination of hyaluronic acid, hyaluronidase inhibitor, and hydroxypropyl tetrahydropyrantriol. The combination of hyaluronic acid, hyaluronidase inhibitor, and hydroxypropyl tetrahydropyrantriol provide moisturizing of the skin upon application and allows the delivery and absorption of hyaluronic acid. Among other beneficial interactions, the hydroxypropyl tetrahydropyrantriol, when applied to the skin, according to the present disclosure, promotes natural hyaluronic acid synthesis in the skin.

Hyaluronic Acid

“Hyaluronic acid”, also known as “hyaluronan” and “hyaluronate”, as used herein, relates to polymers of disaccharides composed of D-glucuronic acid and D-N-acetylglucosamine, linked together via alternating β-1,4 and β-1,3 glycosidic bonds. Hyaluronan can be 25,000 disaccharide repeats in length and range in size from 5,000 (low molecular weight end of range) to 20,000,000 (high molecular weight end of range) Da in vivo. The average molecular weight in human synovial fluid is 3-4 million Da. The hyaluronic acid according to the present disclosure includes salts and derivatives of hyaluronic acid. In some embodiments, a hyaluronic acid may be selected from low molecular weight hyaluronic acid, high molecular weight hyaluronic acid, and hyaluronic acid derivatives selected from the group consisting of hydrolyzed hyaluronic acid and encapsulated hyaluronic acid.

At least one hyaluronic acid will typically be present in the composition in an amount of from about 0.1% to about 1.4%, or from about 1.0% to about 1.3%, or from about 1.1% to about 1.2% by weight or about 1.2%, by weight of the composition. In some embodiments, the composition comprises from about 1.0% to about 1.3% of a combination of two or more hyaluronic acids comprising hyaluronic acid and hyaluronic acid derivatives.

In some embodiments, the composition includes more than one hyaluronate comprising one of a low molecular weight hyaluronic acid and a high molecular weight hyaluronic acid, and one or more hyaluronic acid derivatives selected from the group consisting of hydrolyzed hyaluronic acid and encapsulated hyaluronic acid (for example, cellulose acetate butyrate (and) polyphosphorylcholine glycol acrylate (and) ammonium polyacryloyldimethyl taurate (and) polyvinyl alcohol (and) sodium chloride (and) butylene glycol (and) sodium hyaluronate), each hyaluronic acid present in the composition in a range from about 0.1% to about 3%, by weight of the composition. In some particular embodiments, the composition includes one of a low molecular weight hyaluronic acid and a high molecular weight hyaluronic acid, and at least two hyaluronic acid derivatives comprising hydrolyzed hyaluronic acid and encapsulated hyaluronic acid.

Hyaluronidase Inhibitor

Suitable hyaluronidase inhibitors or anti-hyaluronidase include but are not limited to purple rice extract (e.g., Oryza sativa (rice) extract), dipotassium glycyrrhizate, Cucumis sativus fruit, Terminalia catappa, ascorbic acid, mushroom stem extract, Ganoderma cucidum extract and combinations thereof. In a particularly preferred embodiment, the hyaluronidase inhibitor is purple rice extract (e.g., Oryza sativa (rice) extract) or dipotassium glycyrrhizate.

At least one hyaluronidase inhibitor will typically be present in the composition in an amount of from about 0.1% to about 3.0%, or from about 1.0% to about 3.0%, or from about 1.5% to about 2.5% by weight or about 2.0%, by weight of the composition.

In some embodiments, the composition includes one or a combination of hyaluronidase inhibitors, each present in the composition in a range from about 0.1% to about 3%, by weight of the composition. In some particular embodiments, the one or a combination of hyaluronidase inhibitors comprises purple rice extract (e.g., Oryza sativa (rice) extract) present in a range from about 0.1% to about 3.0%, or from about 0.1% to about 0.5% by weight. In some such embodiments, the purple rice extract (e.g., Oryza sativa (rice) extract) is present in an amount of about 0.2% of the total weight of the composition.

In some embodiments, the composition includes one or a combination of hyaluronidase inhibitors, each present in the composition in a range from about 0.1% to about 3%, by weight of the composition. In some particular embodiments, the one or a combination of hyaluronidase inhibitors comprises dipotassium glycyrrhizate present in a range from about 0.1% to about 3.0%, or from about 1.0% to about 2.5%, or from about 1.5% to about 2.5% by weight. In some such embodiments, the dipotassium glycyrrhizate is present in an amount of about 2.0% of the total weight of the composition.

Hydroxypropyl Tetrahydropyrantriol

Hydroxypropyl tetrahydropyrantriol includes C-glycoside derivatives such as those described in EP 1 345 919, which is incorporated by reference in its entirety, in particular C-β-D-xylopyranoside-2-hydroxypropane sold by the company Chimex under the name Mexoryl SBB®.

At least one hydroxypropyl tetrahydropyrantriol will typically be present in the composition in an amount of from about 1.0% to about 12.0%, or from about 5.0% to about 12.0%, or from about 9.0% to about 11.0% by weight or about 10.0%, by weight of the composition.

Emulsified Gel System

The composition of the present disclosure includes an emulsified gel system including an emulsifier. In addition to the emulsifier, other suitable water phase and oil phase components may be included.

Emulsifier

The composition of the present disclosure includes an emulsifier to form the emulsified gel and provide stability to the emulsified gel system.

Suitable emulsifiers include compounds having a fatty chain in the ester of a fatty acid and an (alkyl)glucose and in the oxyalkylenated ether of a fatty acid ester of an (alkyl)glucose may contain from about 8 to about 30 carbon atoms and more particularly from about 10 to about 22 carbon atoms.

Suitable esters of a fatty acid and glucose or an alkylglucose, include the fatty esters of methylglucoside such as the diester of methylglucoside and oleic acid (CTFA name: methyl glucose dioleate); the mixed ester of methylglucoside and an oleic acid/hydroxystearic acid mixture (CTFA name: methyl glucose dioleate/hydroxystearate); the ester of methylglucoside and isostearic acid (CTFA name: methyl glucose isostearate); the ester of methylglucoside and lauric acid (CTFA name: methyl glucose laurate); the ester of methylglucoside and isostearic acid (CTFA name: methyl glucose isostearate); a mixture of the monoester and diester of methylglucoside and isostearic acid (CTFA name: methyl glucose sesquistearate); a mixture of the monoester and diester of methylglucoside and stearic acid (CTFA name: methyl glucose sesquistearate) and a product marketed under the name Glucate SS by the company Amerchol. Mixtures of these esters are also contemplated.

Preferably, the ester of a fatty acid and glucose or alkylglucose is introduced into the oily phase of the emulsion and in a quantity sufficient to achieve the desired result. This amount may range from about 0.1% to about 10%, preferably from about 1% to about 3%, by weight of the composition.

The oxyalkylenated ether of a fatty acid and glucose or alkylglucose may contain from about 10 to about 100 oxyalkylenated groups (or moles of an alkylene oxide such as ethylene oxide, propylene oxide or mixtures thereof), preferably from about 20 to about 40 oxyalkylenated groups. Suitable ethers of a fatty acid and glucose or alkylglucose, include, for example, oxyethylenated ethers of a fatty acid and methylglucose, such as a polyethylene glycol ether of a diester of methylglucose and stearic acid containing about 20 moles of ethylene oxide (CTFA name: PEG-20 methyl glucose distearate marketed under the name Glucam E-20 distearate by the company Amerchol); a polyethylene glycol ether of a mixture of the monoester and diester of methylglucose and stearic acid containing about 20 moles of ethylene oxide (CTFA name: PEG-20 methyl glucose sesquistearate marketed under the name Glucamate SSE-20 by the company Amerchol and under the name Grillocose PSE-20 by the company Goldschmidt). Mixtures of these oxyalkylenated ethers can be used.

Preferably, the oxyalkylenated ether is introduced into the aqueous phase of the emulsion and in a quantity sufficient to achieve the desired result. The oxyalkylenated ether may be used in an amount of from about 0.4% to about 10%, preferably from about 0.4% to about 1.0% by weight of the composition.

In some particular embodiments, the composition includes PEG-20 methyl glucose sesquistearate present in a range from about 0.1% to about 10%, or from about 0.1% to about 3%, or from about 0.1% to about 1.0%, or from about 0.4% to about 10%, or about 0.4%, relative to the total weight of the composition.

Adjuvants

In a known manner, the composition according to present disclosure may also contain adjuvants that are common in cosmetics, such as humectants, preserving agents, antioxidants, complexing agents, solvents, fragrances, bactericides, odor absorbers, vitamins, moisturizers, self-tanning compounds and antiwrinkle active agents. The amounts of these various adjuvants are those conventionally used in the field under consideration, for example from 0.01% to 20% of the total weight of the composition. Depending on their nature, these adjuvants may be introduced into the fatty phase, into the aqueous phase and/or into lipid vesicles.

Other adjuvants suitable for use with the emulsified gel according the present disclosure include, for example, water-soluble or water-miscible solvents or co-solvents or oil-soluble or oil-miscible solvents or co-solvents. Suitable examples of additives and adjuvants include, but are not limited to, fatty alcohols, fatty amides, alkylene carbonates, glycols, lower alcohols (e.g. ethanol, propanediol), dispersion enhancing agents, polymers, thickening agents, stabilizers, moisturizers, humectants, colorants, fillers, chelating agents, antioxidants (e.g. BHT, tocopherol), essential oils, fragrances, dyes, neutralizing or pH-adjusting agents (e.g., citric acid, triethylamine (TEA) and sodium hydroxide), preservatives, bactericides, conditioning or softening agents (e.g., panthenol and allantoin), extracts, such as botanical extracts, or any other ingredient commonly used in cosmetics for this type of application.

Additives and adjuvants may be present in the compositions in amounts generally ranging from about 0.01% to about 10% by weight. Examples of cosmetic active agents or dermatological active agents include free-radical scavengers, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in any water phase(s) or oil phase(s) that is/are present in the composition (i.e., aqueous and/or fatty (oil) phase).

In some embodiments, the composition includes an hydroxy acid, or a hydroxy acid derivative comprising capryloyl salicylic acid (also known as 2-hydroxy-5-octanoyl benzoic acid, lipohydroxy acid, or LHA) wherein capryloyl salicylic acid may be present in the composition in a range from about 0.01% to about 20%, or from about 0.01% to about 5%, or from about 0.01% to about 0.5% of the total weight of the composition. In some particular embodiments, the capryloyl salicylic acid is present in an amount up to about 0.5% of the total weight of the composition.

In some embodiments, the composition includes one or a combination of water-soluble or water-miscible solvents or co-solvents, or oil-soluble or oil-miscible solvents or co-solvents, each present in the composition in a range from about 0.01% to about 20%, or from about 0.01% to about 10%, or from about 0.01% to about 5% of the total weight of the composition. In some such embodiments, the one or a combination of water-soluble or water-miscible solvents or co-solvents or oil-soluble or oil-miscible solvents or co-solvents comprises one or more of: one or more fatty compounds, one or more silicone-containing compounds, and one or more glycols.

In some embodiments, the composition comprises one or more fatty compounds. Each of the one or more fatty compounds is present in an amount from about 0.01% to about 20%, or from about 0.01% to about 10%, or from about 0.01% to about 5% of the total weight of the composition. In some particular embodiments, the one or more fatty compounds is present at about 3.5% of the total weight of the composition.

In some embodiments, the composition comprises one or more silicone-containing compounds. The one or more silicone-containing compounds may be selected from bis-PEG/PPG-16/16 PEG/PPG-16/16 dimethicone (AND) caprylic/capric triglyceride, and polymethylsilsesquioxane, wherein each is present in an amount up from about 0.01% to about 20%, or from about 0.01% to about 10%, or from about 0.1% to about 5%, or from about 0.5% to about 1.5% of the total weight of the composition. In some particular embodiments, the composition comprises each of In some particular embodiments, each of bis-PEG/PPG-16/16 PEG/PPG-16/16 dimethicone (AND) caprylic/capric triglyceride, and polymethylsilsesquioxane. In some particular embodiments, the one or more silicone-containing compounds is present from about 0.5% to about 1.5% of the total weight of the composition.

In some embodiments, the composition comprises one or more glycols. In some such embodiments, the one or more glycols may comprise glycerin present in an amount up from about 0.01% to about 20%, or from about 0.01% to about 10%, or from about 0.01% to about 5% of the total weight of the composition. In some particular embodiments, the glycerin is present at about 5% of the total weight of the composition.

In some embodiments, the composition includes one or a combination of polymers comprising silicone polymers present in the composition in a range from about 0.01% to about 20%, or from about 10% to about 20%, or from about 15% to about 20% of the total weight of the composition. In some particular embodiments, the polymers comprising silicone polymers are present in an amount about 16% of the total weight of the composition.

In some embodiments, the composition includes one or a combination of thickeners present in the composition in a range from about 0.01% to about 20%, or from about 0.01% to about 5%, or from about 0.1% to about 1.0%, or from about 0.1% to about 0.8% of the total weight of the composition. In some particular embodiments, the thickeners are present in an amount of about 0.6% of the total weight of the composition.

Of course, a person skilled in the art will take care to choose this or these optional additional compounds so that the advantageous properties intrinsically attached to the composition in accordance with the present disclosure are not, or not substantially, detrimentally affected by the envisaged addition or additions.

Needless to say, a person skilled in the art will take care to select this or of these optional additional compound(s), and/or the amount thereof, such that the advantageous properties of the composition according to the present disclosure are not, or are not substantially, adversely affected by the envisaged addition.

The composition according to the present disclosure finds its application in a wide variety of treatments, especially cosmetic treatments, of the keratinous tissue, such as skin, the lips and the hair, including the scalp, especially for treating, protecting or caring for the skin, the lips and/or the hair, and/or for making up the skin and/or the lips. It may also be intended for treating dry skin and/or dry lips, while at the same time delivering moisturization.

Stability

Compositions, according to the present disclosure, include a stable composition. By “stable” and grammatical variations thereof, it is meant that the compositions have maintained an aesthetically homogeneous phase, wherein there is no visually perceptible signs of phase separation, does not show a grainy texture and/or become inhomogeneous over a period of 8 weeks in a temperature storage range of 4° C. to 45° C., with no significant changes in appearance, pH, viscosity, and color.

Pleasing Tactile Feel

Compositions, according to the present disclosure, include a pleasing tactile feel. By “pleasing tactile feel” and grammatical variations thereof, it is meant that the compositions have a texture that is pleasing to the consumer and is substantially free of rough, clingy or sticky feel, and problems of innocuity with respect to the skin.

Method

The composition according to the present disclosure is prepared by combining the water and oil phase ingredients, including the hyaluronic acid delivery component, in a vessel and heating this water phase while mixing additional phases to form a homogeneous phase. The heating includes heating to a temperature from about 25° C. to 50° C. or about 35° C. The phase ingredients were premixed, when appropriate. The time homogenization is performed for a time that does not exceed 2 minutes. If necessary, the pH is adjusted to desired level.

The following examples are intended to further illustrate the present invention. They are not intended to limit the invention in any way. Unless otherwise indicated, all parts are by weight.

### Examples

TABLE 1

| Phase | INCI | Ex. 1 | Ex. 2 | Ex. 3 |
|---|---|---|---|---|
| A1 | WATER | q.s. | q.s. | q.s. |
| | GLYCERIN | 5.00 | 5.00 | 5.00 |
| | ADENOSINE | 0.04 | 0.04 | 0.04 |
| | PEG-20 METHYL GLUCOSE SESQUISTEARATE | 0.40 | 0.40 | 0.5 |
| | PRESERVATIVE | 0.90 | 0.90 | 0.90 |
| | DIPOTASSIUM GLYCYRRHIZATE | 2.00 | 2.00 | 2.00 |
| | CELLULOSE ACETATE BUTYRATE (and) | 0.10 | 0.10 | 0.10 |
| | POLYPHOSPHORYLCHOLINE GLYCOL ACRYLATE (and) | | | |
| | AMMONIUM POLYACRYLOYLDIMETHYL TAURATE | | | |
| | (and) POLYVINYL ALCOHOL (and) SODIUM | | | |
| | CHLORIDE (and) BUTYLENE GLYCOL (and) SODIUM | | | |
| | HYALURONATE | | | |
| | SODIUM HYALURONATE | 1.20 | 1.20 | 1.20 |
| A2 | DEXTRIN (and) ORYZA SATIVA (RICE) EXTRACT | 0.2 | 0.2 | 0.2 |
| B | THICKENER | 0.60 | 0.60 | 0.60 |
| C | HYDROXYPROPYL TETRAHYDROPYRANTRIOL | 10.00 | 10.00 | 10.00 |
| D | WATER | 5.00 | 5.00 | 5.00 |
| | SODIUM HYDROXIDE | 0.045 | 0.045 | 0.045 |
| E | ORGANIC SOLVENT | 5.50 | 5.50 | 6.50 |
| | CAPRYLOYL SALICYLIC ACID | 0.50 | 0.50 | 0.50 |
| | AOX | 0.50 | 0.50 | 0.00 |
| F | SILICONE POLYMER | 16.00 | 16.00 | 16.00 |
| | BIS-PEG/PPG-16/16 PEG/PPG-16/16 DIMETHICONE | 0.50 | 0.50 | 0.50 |
| | (and) CAPRYLIC/CAPRIC TRIGLYCERIDE | | | |
| G | POLYMETHYLSILSESQUIOXANE | 1.50 | 1.50 | 0.00 |
| | LAUROYL LYSINE | 0.00 | 0.00 | 1.00 |
| J | FRAGRANCE | 0.015 | 0.02 | 0.02 |
| | STABILITY | YES | YES | YES |
| | TEXTURE | PASS | PASS | PASS |

TABLE 2

| | | Comp. | Comp. | Comp |
|---|---|---|---|---|
| Phase | INCI | Ex. 4 | Ex. 5 | Ex. 6 |
| A1 | WATER | q.s. | q.s. | q.s. |
| | GLYCERIN | 5.00 | 0 | 7.00 |
| | ADENOSINE | 0.04 | 0.04 | 0.04 |
| | PEG-20 METHYL GLUCOSE SESQUISTEARATE | 0.20 | 0 | 0 |
| | PRESERVATIVE | 1.00 | 0.40 | 1.00 |
| | DEXTRIN (and) ORYZA SATIVA (RICE) EXTRACT | 2.00 | 0 | 0 |
| | CELLULOSE ACETATE BUTYRATE (and) | 0.10 | 0.10 | 0 |
| | POLYPHOSPHORYLCHOLINE GLYCOL | | | |
| | ACRYLATE (and) AMMONIUM | | | |
| | POLYACRYLOYLDIMETHYL TAURATE (and) | | | |
| | POLYVINYL ALCOHOL (and) SODIUM | | | |
| | CHLORIDE (and) BUTYLENE GLYCOL (and) | | | |
| | SODIUM HYALURONATE | | | |
| | SODIUM HYALURONATE | 1.20 | 1.10 | 0.65 |
| B | THICKENER | 0.60 | 0 | 0 |
| C | HYDROXYPROPYL TETRAHYDROPYRANTRIOL | 10.00 | 30.00 | 10.00 |
| | LUARETH-23 | 0 | 2.50 | 0 |
| | AOX | 0.50 | 0.50 | 0 |
| | PENTAERYTHRITYL TETRA-DI-T-BUTYL | 0 | 0.10 | 0 |
| | HYDROXYHYDROCINNAMATE | | | |
| | CAPRYLOYL SALICYLIC ACID | 0 | 0.30 | 0 |
| D | WATER | 5.00 | 15.00 | 0 |
| | NIACINAMIDE | 0 | 1.00 | 0 |
| | CAFFEINE | 0 | 0.40 | 0 |
| | DEXTRIN (and) ORYZA SATIVA (RICE) EXTRACT | 0 | 0.20 | 0.50 |
| | DIPOTASSIUM GLYCYRRHIZATE | 0 | 2.00 | 0.50 |
| | SODIUM HYDROXIDE | 0.045 | 0 | 0 |
| | PRESERVATIVE | 0 | 0.10 | 0 |
| E | ORGANIC SOLVENT | 5.50 | 0 | 10.00 |
| | CAPRYLOYL SALICYLIC ACID | 0.50 | 0 | 0 |
| F | SILICONE POLYMER | 16.00 | 0 | 0 |
| | BIS-PEG/PPG-16/16 PEG/PPG-16/16 | 0.50 | 0 | 0 |
| | DIMETHICONE (and) CAPRYLIC/CAPRIC | | | |
| | TRIGLYCERIDE | | | |
| G | POLYMETHYLSILSESQUIOXANE | 1.50 | 0 | 0 |
| H | FRAGRANCE | 0.02 | 0 | 0.02 |
| | STABILITY | NO | NO | YES |
| | TEXTURE | PASS | PASS | FAIL |

The compositions shown in Tables 1 and 2 were prepared by heating water phase ingredients, including those shown as A1 and A2 (as shown in Tables 1 and 2) to about 35°. Additional phases are added to the heated composition and mixing is performed until the composition is homogenized. The phase ingredients were premixed, when appropriate. The time homogenization is performed for a time that does not exceed 2 minutes. If necessary, the pH is adjusted to desired level (5.25+/−0.25).

Thereafter, the composition was cooled to room temperature. Stability was evaluated “NO” denotes incompatibility, indicating that the emulsion destabilizes and the composition separated. “YES” denotes that the composition maintained stability. In addition, the composition was evaluated for texture. The composition was also evaluated for texture to determine stability. The composition was applied to the skin to determine whether the composition is free of a rough, clingy or sticky feel, and problems of innocuity with respect to the skin. An indication of “PASS” indicated that the emulsion had a pleasing tactile feel and “FAIL” indicated that emulsion did not have a pleasing tactile feel.

Example 2 was evaluated for clinical effectiveness. The results of the testing are shown in Table 3.

TABLE 3

| Clinical Grading | | Example 2 | | |
|---|---|---|---|---|
| Assessment | Location | Week 4 | Week 8 | Week 12 |
| Crepiness | Neck | X | XX | XX |
| Firmness (tactile) | Global face | X | XX | XX |
| Overall skin | Global face | X | XX | XX |
| appearance/quality | | | | |
| Radiance | Global face | XX | XX | XX |
| Skin density | Global face | X | XX | XX |
| Skin elasticity | Global face | X | XX | XX |
| Skin plumpness | Global face | XX | XX | XX |
| Skin sagginess | Global face | X | XX | XX |
| Skin texture (tactile) | Global face | XX | XX | XX |
| | Neck | XX | XX | XX |
| Skin tone evenness | Global face | X | X | XX |
| Fine lines/Wrinkles | Crow's feet | X | XX | XX |
| | Nasolabial fold | X | X | XX |
| | Marionette lines | X | XX | XX |

XX indicates statistical significant p-value ≤ 0.05 and clinical significance (mean change ≤ −0.26)
X indicates statistically significant improvement when compared to baseline (p ≤ 0.05).

Based on clinical expert grading, the composition of Example 2 showed a statistically significant decrease (improvement) in all attributes for all time points assessed.

After Week 4 of product use, the composition of Example 2, showed a significant improvement in the following attributes:
Crepiness (neck)
Firmness
Overall skin appearance/quality
Radiance
Skin density
Skin elasticity
Skin plumpness
Skin sagginess
Skin texture tactile (face and neck)
Skin tone evenness
Fine lines/wrinkles (crow's feet, nasolabial fold, marionette lines)

Additionally, radiance, skin plumpness, and skin texture tactile (face and neck) showed clinical significance at Week 4.

After Week 8 of product use, the composition of Example 2, showed a significant improvement in the following attributes:
Crepiness (neck)
Firmness
Overall skin appearance/quality
Radiance
Skin density
Skin elasticity
Skin plumpness
Skin sagginess
Skin texture tactile (face and neck)
Skin tone evenness
Fine lines/wrinkles (crow's feet, nasolabial fold, marionette lines)

Additionally, crepiness, firmness, overall skin appearance/quality, radiance, skin density, elasticity, plumpness, sagginess, skin texture tactile (face and neck) and fine lines/wrinkles (crow's feet and marionette lines) showed clinical significance at Week 8.

After Week 12 of product use, the composition of Example 2, showed a significant improvement in the following attributes:
Crepiness (neck)
Firmness
Overall skin appearance/quality
Radiance
Skin density
Skin elasticity
Skin plumpness
Skin sagginess
Skin texture tactile (face and neck)
Skin tone evenness
Fine lines/wrinkles (crow's feet, nasolabial fold, marionette lines)

Additionally, all the assessed clinical attributes showed clinical significance at Week 12.

Based on Corneometer measurements, there was a statistical significant increase in Corneometer measurements at Week 12 when compared to baseline, indicating an improvement in hydration after 12 weeks of product use.

Based on the TEWL measurements, there was a statistical significant decrease in TEWL measurements at Week 12 when compared to baseline, indicating an improvement in barrier function after 12 weeks of product use.

Based on the ultrasound measurements, there was a statistical significant increase in skin density at Week 12 when compared to baseline. However, there were no statistical differences in skin thickness.

Based on raking light image analysis, there was a statistical significant decrease in the area (in mm²) of the crow's feet. Although not statistically significant, the number, length, width and depth showed directional decrease (improvement/less severe) in wrinkles.

Based on tolerance grading, the composition of Example 2, objective and subjective tolerance evaluations showed no statistically significant differences when compared to baseline except for dryness which showed improvement at Weeks 4, 8 and 12, and erythema at Week 8.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. An emulsified gel composition, the gel composition comprising:
an emulsified gel system comprising:
(1) a hyaluronic acid delivery component, the hyaluronic acid delivery component comprising a combination of active ingredients including each of:
(a) hyaluronic acid present in an amount from about 1.0% to about 1.3%, by weight of the composition;
(b) hyaluronidase inhibitor comprising dipotassium glycyrrhizate, present in an amount from about 1.5% to about 2.5%, by weight of the composition, and purple rice extract, present in an amount from about 0.1% to about 0.5%, by weight of the composition; and
(c) hydroxypropyl tetrahydropyrantriol present in an amount from about 5.0% to about 12.0%, by weight of the composition; and
(2) an emulsifier present in an amount from about 0.1% to about 1.0%, by weight of the composition,
wherein the composition exhibits stability over a period of 8 weeks storage in a temperature storage range of 4° C. to 45° C. which stability is characterized by the absence of visually perceptible signs of phase separation.

2. The composition of claim 1, further comprising a hydroxy acid present in the composition in a range from about 0.01% to about 20%, by weight of the composition.

3. The composition of claim 1, further comprising a hydroxy acid derivative comprising capryloyl salicylic acid present wherein the capryloyl salicylic acid is present in a range from about from about 0.01% to about 0.5% of the total weight of the composition.

4. The composition of claim 1, wherein the hyaluronic acid comprises a hyaluronic acid selected from the group consisting of a low molecular weight hyaluronic acid, a high molecular weight hyaluronic acid, a hyaluronic acid derivative, and combinations thereof.

5. The composition of claim 4, wherein the hyaluronic acid derivative is selected from the group consisting of hydrolyzed hyaluronic acid and encapsulated hyaluronic acid.

6. The composition of claim 1, wherein the hyaluronic acid comprises each of a low molecular weight hyaluronic acid, a hyaluronic acid derivative comprising hydrolyzed hyaluronic acid, and a hyaluronic acid derivative comprising an encapsulated hyaluronic acid cellulose acetate butyrate (and) polyphosphorylcholine glycol acrylate (and) ammonium polyacryloyldimethyl taurate (and) polyvinyl alcohol (and) sodium chloride (and) butylene glycol (and) sodium hyaluronate), wherein each hyaluronic acid is present in the composition in the composition a range from about 0.1% to about 1.3%, by weight of the composition.

7. The composition of claim 1, wherein the hydroxypropyl tetrahydropyrantriol is present in an amount from about from about 9.0% to about 11.0%, by weight of the composition.

8. The composition of claim 1, wherein the emulsifier is an oxyalkylenated ether of a fatty acid and glucose or alkylglucose.

9. The composition of claim 8, wherein the emulsifier is PEG-20 methyl glucose sesquistearate.

10. The composition of claim 1, further comprising one or more compounds selected from one or more fatty compounds, one or more silicone-containing compounds, one or more glycols and combinations thereof, each present in the composition in a range from about 0.01% to about 20%.

11. The composition of claim 1, further comprising one or more compounds selected from (i) one or more fatty compounds, each present in an amount up from about 0.01% to about 5% of the total weight of the composition, (ii) one or more silicone-containing compounds, each present in an amount up from about 0.5% to about 1.5% of the total weight of the composition, (iii) one or more glycols, each present in an amount from about 0.01% to about 10%, and (iv) combinations thereof.

12. The composition of claim 11, comprising one or more silicone-containing compounds selected from the group consisting of bis-PEG/PPG-16/16 PEG/PPG-16/16 dimethicone (AND) caprylic/capric triglyceride, and polymethylsilsesquioxane, and combinations thereof, and comprising one or more glycols comprising glycerin.

13. The composition of claim 1, wherein the stability is characterized by the absence of visually perceptible signs of changes in pH, viscosity, and color.

14. The composition of claim 1, wherein the emulsified gel system is an oil in water emulsion.

15. The emulsified gel composition of claim 3, wherein: the dipotassium glycyrrhizate is present at about 2.0%, by weight of the composition; the purple rice extract is present at about 0.2%, by weight of the composition; the hydroxypropyl tetrahydropyrantriol is present at about 10.0%, by weight of the composition; the emulsifier is present at about 0.4%, by weight of the composition; and the capryloyl salicylic acid is present at about 0.5%, by weight of the composition.

16. An emulsified gel composition, the gel composition comprising:
an emulsified gel system comprising an emulsifier comprising an oxyalkylenated ether of a fatty acid and glucose or alkylglucose, capryloyl salicylic acid, and a hyaluronic acid delivery component comprising:
(a) hyaluronic acid;
(b) a combination of hyaluronidase inhibitors, the combination comprising dipotassium glycyrrhizate and purple rice extract; and
(c) hydroxypropyl tetrahydropyrantriol,
wherein the composition exhibits stability over a period of 8 weeks storage in a temperature storage range of 4° C. to 45° C. which stability is characterized by the absence of visually perceptible signs of phase separation.

17. The composition of claim 16, wherein the hyaluronic acid comprises each of a low molecular weight hyaluronic acid, a hyaluronic acid derivative comprising hydrolyzed hyaluronic acid, and a hyaluronic acid derivative comprising an encapsulated hyaluronic acid cellulose acetate butyrate (and) polyphosphorylcholine glycol acrylate (and) ammonium polyacryloyldimethyl taurate (and) polyvinyl alcohol (and) sodium chloride (and) butylene glycol (and) sodium hyaluronate), wherein each hyaluronic acid is present in the composition in the composition a range from about 0.1% to about 1.3%, by weight of the composition.

18. The emulsified gel composition of claim 17, wherein: the dipotassium glycyrrhizate is present in an amount from about 1.5% to about 2.5%, by weight of the composition; the purple rice extract is present in an amount from about 1.5% to about 2.5%, by weight of the composition; the hydroxypropyl tetrahydropyrantriol is present in an amount from about 5.0% to about 12.0%, by weight of the composition; the emulsifier is present in an amount from about 0.4% to about 1.0%, by weight of the composition; and the capryloyl salicylic acid is present in a range from about from about 0.01% to about 0.5% of the total weight of the composition.

19. The emulsified gel composition of claim 17, wherein: the dipotassium glycyrrhizate is present at about 2.0%, by weight of the composition; the purple rice extract is present at about 0.2%, by weight of the composition; the hydroxypropyl tetrahydropyrantriol is present at about 10.0%, by weight of the composition; the emulsifier is present at about 0.4%, by weight of the composition; and the capryloyl salicylic acid is present at about 0.5%, by weight of the composition.

20. A method for cosmetic treatment of keratinous tissues by applying onto the keratinous tissue a composition comprising:
an emulsified gel system comprising an emulsifier comprising an oxyalkylenated ether of a fatty acid and glucose or alkylglucose, capryloyl salicylic acid, and a hyaluronic acid delivery component comprising:
(a) hyaluronic acid;
(b) a combination of hyaluronidase inhibitors, the combination comprising dipotassium glycyrrhizate and purple rice extract; and
(c) hydroxypropyl tetrahydropyrantriol,
wherein the composition exhibits stability over a period of 8 weeks storage in a temperature storage range of 4° C. to 45° C. which stability is characterized by the absence of visually perceptible signs of phase separation.
